(19) United States
(12) Patent Application Publication
(10) Pub. No.: US 2005/0096637 A1
(43) Pub. Date: May 5, 2005

(54) Sensing food intake

(75) Inventor: Kenneth T. Heruth, Edina, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/698,124

(22) Filed: Oct. 31, 2003

(51) Int. Cl. A61K 9/22

(52) U.S. Cl. 604/891.1

(57) ABSTRACT

The invention provides methods and devices to sense food intake, and parameters such as glucose concentration that change as a function of food intake, in a patient. The invention provides for measurement of physiological parameters that change as a function of food intake, such as a core body temperature. Some embodiments of the invention also measure activity of the patient. The invention may further provide methods and devices to provide treatment for medical conditions related to food intake, such as delivery of insulin or other substance to regulate the blood glucose of the patient.

### FIELD OF THE INVENTION

The invention relates to sensing food intake, and, more particularly, sensing food intake as a function of physiological parameters.

### BACKGROUND

Food intake for a patient is pertinent to a variety of medical conditions, such as diabetes, obesity, and bulimia. In general, treatment for such conditions can include controlling the diet and monitoring delivery of medication. A patient having Type I diabetes, for example, may manage the disorder by maintaining a strict diet and by ingesting or injecting medication to regulate blood glucose levels. A patient having Type II diabetes, by contrast, may manage the disorder principally by monitoring the diet.

Diabetes is a disease in which the body does not produce an adequate amount of insulin, or does not respond properly to the insulin produced resulting in an accumulation of glucose in the blood (hyperglycemia). Blood glucose is affected by many factors including the quantity of food ingested, the type of food ingested, exercise, stress, illness and the like. Allowing the glucose level to be too high can result in ketoacidosis (diabetic coma) and vascular complications with long-term effects such as damage to the retinas, kidneys, nerves and blood vessels. However, a low glucose level, (hypoglycemia) may cause loss of consciousness, seizures, neurological deficit, and death. Many diabetics monitor their blood glucose several times a day to maintain a tight control of the blood glucose level.

Health risks associated with obesity are well known. An obese patient is at increased risk of high blood pressure, heart disease, stroke, high cholesterol, breathing problems, sleep apnea, cancer, gallstones, and arthritis, among other health problems. An obese patient is also at increased risk of developing Type II diabetes. Similarly, the health risks associated with bulimia and other eating disorders are well known.

For a patient having diabetes, obesity, an eating disorder or another condition, it is helpful to monitor the quantity of food ingested by the patient. In some instances, the patient develops the discipline to keep a journal of foods consumed, but in some circumstances, the patient does not know this information or could benefit from additional information. Food intake for a patient can be measured by a number of techniques, including direct measurement of the contents of the stomach. Because food intake triggers numerous physiological responses in the body, food intake can be monitored by measuring or monitoring physiological parameters that change as a function of food intake.

In addition, some diabetics monitor blood glucose using various techniques. Some diabetics manually check blood glucose levels at certain times. Others rely upon implanted sensors that apply electrochemical methods such as the electroenzymatic method where blood glucose is oxidized under glucose-oxidase control, producing gluconic acid and hydrogen peroxide. Long-term monitoring systems and devices known in the art involve chemically based sensors that are generally replaced periodically. Examples of these techniques and/or devices may be found in the issued U.S. Patents listed in Table 1 below.

TABLE 1

| Patent Number | Inventors | Title |
|---|---|---|
| 4,003,379 | Ellinwood, | Apparatus and method for implanted |
| | Jr. | self-powered medication dispensing |
| 6,508,762 | Karnieli | Method for monitoring food intake |
| 5,563,850 | Hanapole | Food intake timer |
| 5,398,688 | Laniado | Method, system and instrument for |
| | | monitoring food intake |
| 4,221,959 | Sessler | Checking device for checking the |
| | | food intake |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY OF THE INVENTION

The present invention has certain objects. That is, various embodiments of the present invention provide solutions to one or more problems existing in the prior art with respect to prior techniques for sensing food intake. These problems include the medical and economic benefit associated with implanting known, reliable, long life sensors in a patient that respond to food intake. For example, current implantable sensors to measure blood glucose for the treatment of diabetes have a comparatively short useful life and are generally replaced periodically. Further, manually checking blood glucose levels at recommended times is not always possible. Relying on physical symptoms to indicate need of treatment is unreliable and very hazardous to the health of the diabetic. The problems also include a patient's inability, for any number of reasons, to monitor food intake. In addition, the problems include difficulties associated with providing treatment to the patient based on food intake. Various embodiments of the present invention have the object of solving at least one of the foregoing problems.

The present invention includes features to measure physiological parameters that change as a function of food intake via implanted reliable long life sensors and features to estimate the quantity of food consumed by the patient based on the measurement. Physiological parameters, such as core body temperature, enlargement of the gastrointestinal tract, electrical activity of the gastrointestinal tract, transabdominal impedance and the like, vary with food intake. The present invention includes a processor that, in some embodiments, estimates blood glucose as a function of food intake. Various embodiments also include a feature to measure the activity of the patient, which also may affect blood glucose levels.

An additional feature of the present invention can deliver therapy to the patient as a function of the estimated food intake. In one embodiment of the invention, one or more drugs may be delivered to the patient as a function of the sensed food intake. Patients having an implantable drug delivery device may receive, for example, insulin via a first drug pump and glucagon via a second drug pump to regulate blood glucose. By monitoring the physiological parameters alone or in combination, an implanted drug delivery device can more responsively and therapeutically administer drugs to the patient.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a diagram illustrating a system for measuring physiological parameters that change as a function of food intake.

FIG. 2 is a block diagram illustrating constituent components of an embodiment of the invention depicted in FIG. 1.

FIG. 3 is a flow diagram illustrating a technique for sensing food intake and providing treatment for diabetes.

FIG. 4 is a flow diagram illustrating a technique for sensing activity level and providing treatment for diabetes.

### DETAILED DESCRIPTION OF THE PREFERRED EMBODIMENTS

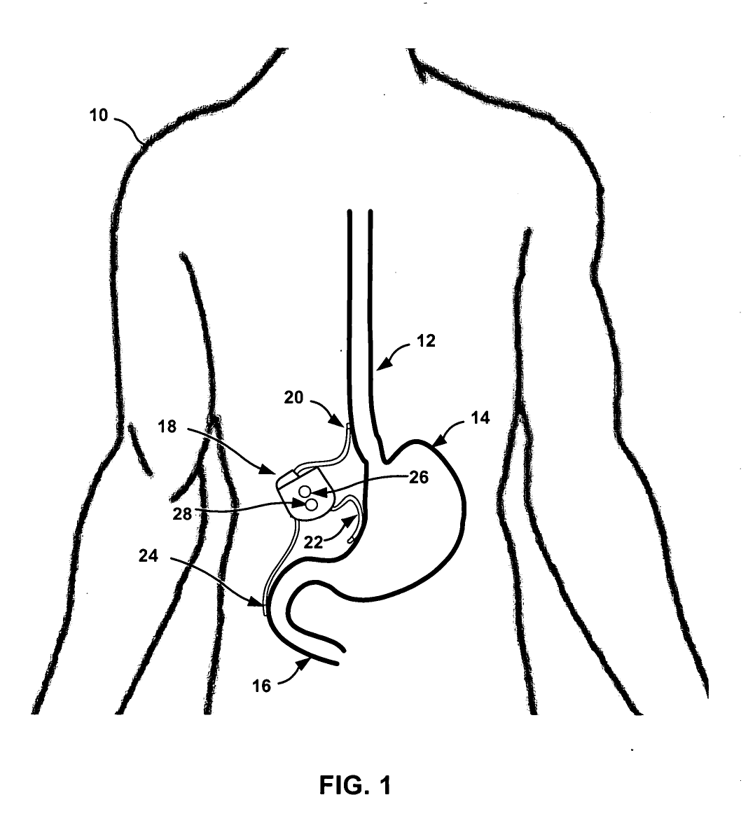

FIG. 1 is a block diagram illustrating a view of the gastro-intestinal tract of a patient 10, in which esophagus 12, stomach 14, and a portion of small intestine 16 are visible. FIG. 1 illustrates devices for measuring physiological parameters that change as a function of food intake and estimating the quantity of food consumed based on the measurement.

Implantable medical device (“IMD”) 18 may be any of several implantable devices. IMD 18 may take the form of a gastro-intestinal pacemaker, for example, or a drug delivery device. Examples of implantable gastro-intestinal pacemakers and tract stimulators include devices disclosed in U.S. Pat. No. 6,243,607 to Mintchev et al., U.S. Pat. No. 5,690,691 to Chen et al., or U.S. Pat. No. 6,453,199 to Kobozev all hereby incorporated by reference herein, each in its respective entirety. An example of an implantable drug delivery system includes a number of SynchroMed pumps manufactured by and commercially available from Medtronic Inc.

In the exemplary embodiment depicted in FIG. 1, three electrodes (20, 22, and 24) are deployed on or proximate to esophagus 12, stomach 14, and intestine 16 respectively, and are coupled by leads to IMD 18. Electrodes 20, 22, and 24 operate as sensors, detecting signals reflecting electrical activity of the gastro-intestinal tract. Electrodes 20, 22, and 24 may further deliver pacing pulses generated by IMD 18 to the gastro-intestinal tract, thereby electrically stimulating the smooth muscle. In some embodiments of the invention, electrodes 20, 22, and 24 may be supplemented or supplanted with other sensors that respond to the activity of the gastro-intestinal tract, such as motion sensors that sense peristaltic motion.

IMD 18 receives signals detected via electrodes 20, 22, and 24 that reflect electrical activity of the gastro-intestinal tract. IMD 18 processes the signals to determine the quantity of food consumed by patient 10. Signal processing includes, but is not limited to, measurement and analysis of timing and duration of the detected signals. In response to the detected signals, IMD 18 can estimate the quantity of food consumed by patient 10 in a meal. IMD 18 can further estimate the caloric content of the food consumed by patient 10 in the meal.

IMD 18 may take into account factors for estimation of caloric content of a meal other than or in addition to signals detected via electrodes 20, 22, and 24. For example, IMD 18 may take into account whether patient 10 follows a regulated diet, and IMD 18 may estimate the calories consumed according to the diet. A patient suffering from Type I diabetes, for example, typically follows a strict diet to regulate blood glucose. In such a patient, IMD 18 may estimate the quantity of ingested glucose as a function of the quantity of food ingested by patient 10. The mathematical relationship between sensed physiological parameters and the estimated quantity of food ingested may be derived empirically, and may vary from patient to patient.

When IMD 18 comprises a drug delivery system, IMD 18 may control delivery of drugs as a function of signals detected via electrodes 20, 22, and 24. As described below, IMD 18 can monitor one or more physiological parameters that change as a function of food consumed by a patient and deliver drug therapy as a function of the monitored parameters. IMD 18 can also estimate caloric content, glucose concentration or other characteristics as a function of the quantity of food consumed. When embodied as a drug delivery system, IMD 18 can, for example, control blood glucose with drug delivery, thereby resulting in less risk of hyperglycemia or hypoglycemia or both.

IMD 18 may process other signals reflecting physiological parameters that change as a function of food intake. For example, IMD 18 may be coupled to an electrode pair (not shown) that supplies signals to IMD 18 as a function of transabdoimnal impedance. Such an electrode pair may include a first electrode located anterior to stomach 14 and a second electrode located posterior to stomach 14. The transabdominal impedance signal varies as patient 10 consumes food and stomach 14 fills and enlarges. IMD 18 processes the signal and estimates the quantity of food consumed as a function of the transabdominal impedance signal. IMD 18 may also estimate caloric content, glucose concentration or other characteristics as a function of the transabdominal impedance signal.

Another physiological parameter of interest is core body temperature. IMD 18 may monitor core body temperature via a temperature sensor 26. As depicted in FIG. 1, temperature sensor 26 is bonded to the housing of IMD 18. Temperature sensor 26 may also be deployed proximate to esophagus 12, stomach 14, or intestine 16, or other internal organ of patient 10, and coupled to IMD 18 with a lead.

In general, core body temperature of patient 10 decreases as food is digested, and the change in core body temperature is a function of the quantity of food ingested. Though the change in core body temperature following a meal is on the order of a fraction of a degree Celsius, but the change is detectable and of clinical significance. IMD 18 receives the temperature signal from temperature sensor 26 and estimates the quantity of food consumed by patient 10 as a function of the temperature signal.

In some embodiments of the invention, IMD 18 estimates food intake, caloric content, glucose concentration or other characteristics as a function of signals from a single sensor. In other embodiments of the invention, IMD 18 performs the estimation as a function of signals from multiple sensors.

IMD 18 may further include an activity sensor 28 inside or coupled to the housing of IMD 18, or separately implanted in the abdomen of patient 10. Activity sensor 28 may take the form of an accelerometer that responds to the physical motion of patient 10. Activity sensor 28 may also be any other sensor that generates a signal that varies as a function of a measured parameter relating to metabolic requirements of a patient. For example, activity sensor 28 may include a sensor that responds to the heart rate of patient 10.

The quantity and type of food ingested by patient 10 is not the only factor that bears upon the amount of glucose in the blood of patient 10. The activity of patient 10 also has an effect. When IMD 18 comprises a drug delivery system, IMD 18 may control delivery of drugs as a function of patient activity. In some embodiments of the invention, IMD 18 may administer one drug in response to food intake, caloric content, glucose concentration or other characteristics, and another drug in response to patient activity. In this way, IMD 18 may embody a closed loop insulin/glucagon delivery system that delivers an appropriate drug to patient 10 when detected conditions indicate that such drug delivery is appropriate, and that monitors the response of patient 10 to the delivery of the drug. In one implementation, IMD 18 may deliver insulin or glucagon to regulate the glucose level of patient 10.

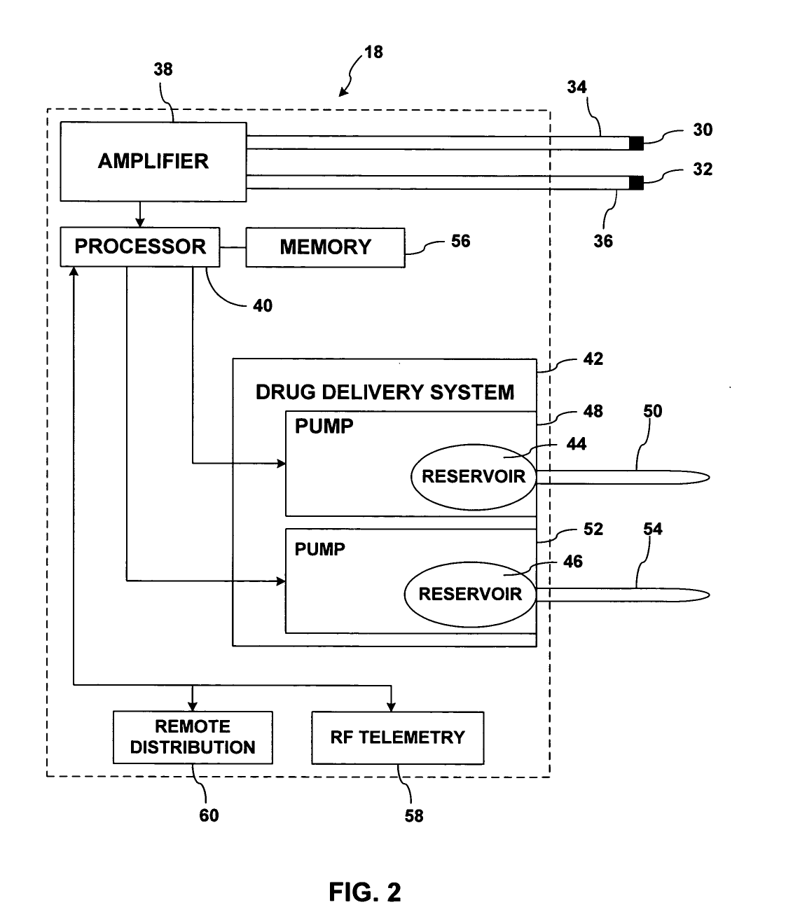

FIG. 2 is a block diagram illustrating an embodiment of the invention. In FIG. 2, IMD 18 comprises a drug delivery system configured to deliver insulin or glucagon to patient 10. In FIG. 2, IMD 18 is coupled to two sensors 30, 32 by leads 34, 36. Sensors 30, 32 may be any of several sensors that detect physiological parameters, such as a temperature sensor, a transabdominal impedance sensor or an activity sensor. In some embodiments of the invention, leads may be coupled to IMD 18 that include electrodes to administer therapy, such as electrodes to electrically stimulate the gastrointestinal tract to enhance or accelerate peristaltic movement.

An amplifier in IMD 18 may receive signals detected by sensors 30, 32. Amplifier 38 amplifies and filters the received signals and supplies the signals to a processor 40. Processor 40 processes the received signals. Processor 40 estimates the quantity of food consumed by patient 10 as a function of the signals. Processor 40 may also estimate the caloric content of a meal, glucose concentration or other characteristics as a function of the signals. Processor 40 further regulates drug delivery system 42 as a function of the signals. In particular, processor 40 generates one or more control signals that direct drug delivery system 42 to deliver one or more agents to patient 10.

Drug delivery system 42, as depicted in FIG. 2, is configured to deliver two distinct drugs to patient 10 in response to control signals from processor 40, and includes separately controlled apparatus for delivery of each drug. For purpose of illustration, a first reservoir 44 holds insulin and a second reservoir 46 holds glucagon. The agents held by reservoirs 44 and 46 may be selected by the patient's physician, based upon the patient's particular needs. Pump 48 dispenses insulin from reservoir 44 to the patient's body via catheter 50 in response to a control signal from processor 40. Similarly, pump 52 dispenses glucagon from reservoir 46 to the patient's body via catheter 54 in response to a control signal from processor 40.

Reservoirs 44 and 46 are self-sealing and may be refilled by a needle and syringe. Advantageously, drug delivery system 42 need not be surgically removed when reservoirs 44 or 46 are empty. Pumps 48, 52 may further include a fill port (not shown) for refilling the reservoir. Infusion apparatus, such as catheters 50 and 54, infuse drugs from reservoirs 44 and 46 to one or more infusion sites the body. The infusion site may depend upon the drug being infused.

Processor 40 is typically programmable, with programmed instructions residing in memory 56. Memory 56 may include any form of volatile memory, non-volatile memory, or both. In addition, memory 56 may store records concerning measurements of detected physiological parameters, drug deliveries or other information pertaining to operation of IMD 18. Memory 56 may also store information about a regulated diet specified for patient 10, or other data or instructions from a physician for patient 10, such as minimum or maximum dosages, frequency of administration, and the like. The physician may supply data or instructions to IMD 18, or may extract data from IMD 18, via one or more communication links. FIG. 2 shows two exemplary communication links. An RF telemetry link 58 may be used for communication with IMD 18 locally, e.g., when patient 10 is at the office of the physician. Remote distribution link 136 provides a channel for communicating with IMD 18 from a remote location, such as over a telephone line or over the Internet, for example. The invention includes embodiments having other kinds of communication links as well, such as an audible, tactile or radio-controlled alarm module.

In a typical operation, IMD 18 receives signals from sensors 30, 32, and estimates a blood glucose concentration, for example, as a function of the received signals. When the estimated blood glucose concentration indicates hyperglycemia, processor 40 may control pump 48 to deliver insulin from reservoir 44. Pump 48 dispenses insulin from reservoir 44 via catheter 50. IMD 18 continues to receive signals from sensors 30, 32, and estimates a blood glucose concentration as a function of the received signals. In this way, IMD 18 monitors the response of patient 10 to the administered therapy. Similarly, when processor 40 determines that blood glucose is low as a function of signals from sensors 30, 32, processor 40 may control pump 52 to dispense glucagon from reservoir 46 via catheter 54, and may monitor the response of patient 10 to such therapy.

The example of FIG. 2 is offered for purposes of illustration, and the invention is not limited to the circumstances described. The invention is not limited, for example, to an IMD that includes a drug pump, or to a drug pump that dispenses two agents, or to a drug pump that delivers insulin and glucagon. Various embodiments may include, for example, an IMD that administers electrical stimulation in place of or in concert with delivery of drugs.

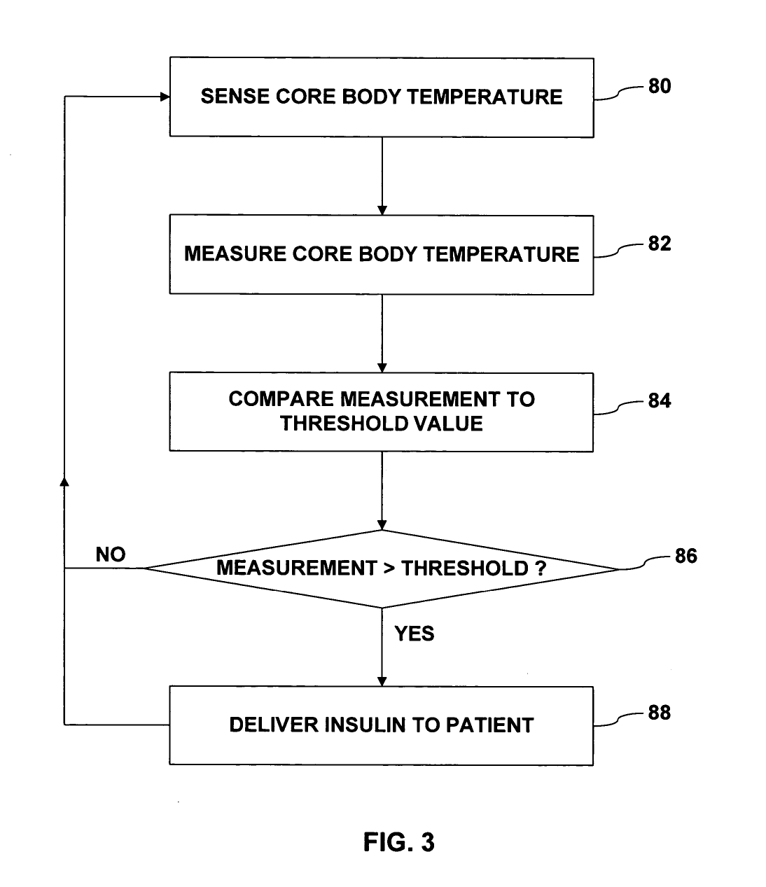

Furthermore, the arrangement of components in FIG. 2 is exemplary. In one embodiment, drug delivery system may be distinct from IMD 18. In that embodiment, processor 40 may be housed inside IMD 18 or housed in the distinct drug delivery system. FIG. 3 is a flow diagram illustrating a technique for treating diabetes by sensing a core body temperature and delivering insulin in response to the sensed core body temperature. Core body temperature decreases during digestion and may reflect food intake by patient 10. Core body temperature may also indicate caloric content of a meal, glucose concentration or other characteristics. Because many diabetics, particularly those suffering from Type I diabetes, follow a strict dietary regimen, processor 40 may estimate the glucose in the food as a function of the quantity of the food consumed. Processor 40 of IMD 18 may regulate insulin delivery as a function of core body temperature. In a typical implementation, processor 40 would be unlikely to regulate insulin delivery solely as a function of core body temperature, but core body temperature is one of a plurality of sensed physiological parameters evaluated by processor 40.

IMD 18 senses core body temperature via temperature sensor 26 (80) and measures core body temperature (82). Measurement of core body temperature (82) may include measurement of an absolute body temperature, measurement of the amplitude of a temperature change, measurement of the rate of temperature change, and so forth. Any one or more of these measurements may be deemed to be of clinical significance. Processor 40 compares the measured temperature to a threshold stored in memory 56 (84). Whether processor 40 will control drug delivery device 42 to deliver insulin depends upon whether the measurement surpasses the threshold.

For example, when the measurement is the amplitude of the core body temperature change, and the amplitude is greater than a threshold stored in memory 56 (86), processor 40 may determine that patient 10 has consumed a large meal, which may result in high blood glucose. In response, processor 40 generates a control signal to control drug delivery device 42 to deliver insulin to patient 10 (88). Processor 40 may continue to monitor the core body temperature (80), or other physiological parameters, to assess the response of patient 10 to the delivery of insulin. When the amplitude of temperature change does not exceed the threshold, monitoring may be continued (80).

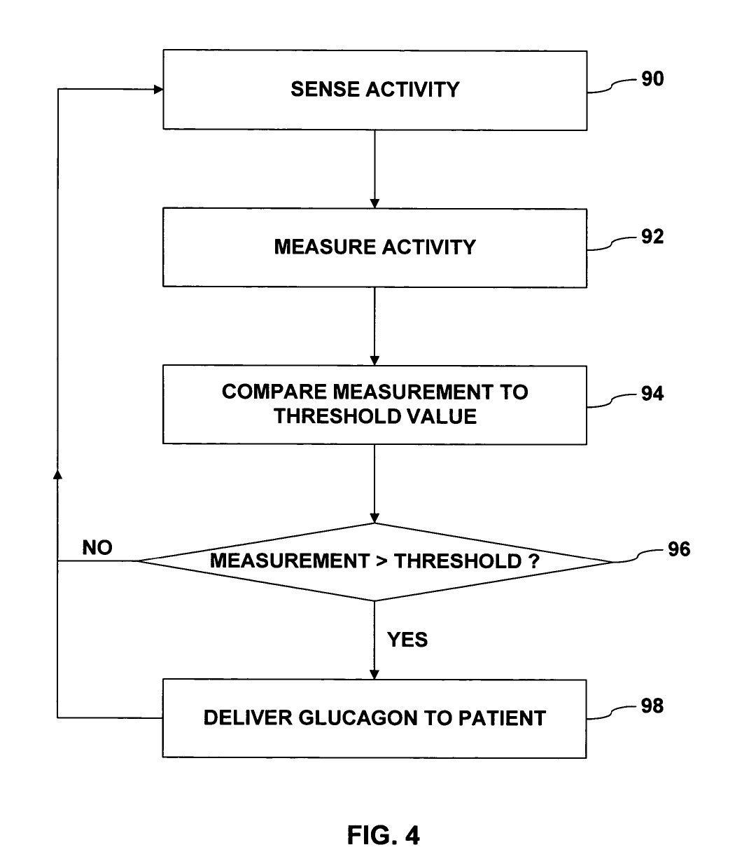

FIG. 4 is a flow diagram illustrating a technique for regulating blood glucose by sensing the activity level of patient 10. In general, a high activity level due to causes a decrease in the amount of glucose in the blood. The activity level of the patient may change when the patient exercises, climbs stairs, goes for a walk, or engages in other physical activity.

Activity sensor 28 senses an activity level (90) by, for example, sensing physical motion of patient 10 or monitoring an increase in the heart rate of patient 10 that accompanies physical activity. IMD 18 measures the activity as a function of signals from activity sensor 28 (92). Measurement of the activity (92) may include, for example, measuring the duration of the activity, the strenuousness of the activity, the number of calories consumed in the activity and so forth. Processor 40 compares the measured activity to a threshold stored in memory 56 (94). Whether processor 40 will control drug delivery device 42 to deliver glucagon depends upon whether the measurement surpasses the threshold.

For example, when the measurement is the number of calories consumed, and the amplitude is greater than a threshold stored in memory 56 (96), processor 40 may determine that patient 10 has engaged in activity to a degree that the blood glucose may be low. In response, processor 40 generates a control signal to control drug delivery device 42 to deliver glucagon to patient 10 (98). Processor 40 may continue to monitor activity (90) or other physiological parameters, to assess the response of patient 10 to the delivery of glucagon. When the activity measurement does not surpass the threshold, monitoring may be continued (90).

Processor 40 may take other action in response to sensed physical activity, such as reduction of insulin delivery to patient 10. Further, processor 40 may take into consideration factors in addition to activity, such as insulin delivery and food intake. In general, processor 40 will control drug delivery device 42 to deliver glucagon when physical activity, food intake and insulin delivery do not produce enough of the desired effect on blood glucose.

In the embodiments depicted in FIGS. 3 and 4, processor 40 compares a measured parameter, such as core body temperature or physical motion of patient 10, to a threshold. The invention also encompasses embodiments in which processor 40 compares an estimated quantity that varies as a function of a measured parameter to a threshold. For example, processor 40 may compare food intake or glucose concentration to a threshold, and each estimate may be a function of a plurality of measurements. In some implementations, a measurement or estimation will “surpass” a threshold when the measurement or estimation is above the threshold, and in other implementations, the measurement or estimation will “surpass” a threshold when the measurement or estimation is below the threshold.

The invention further encompasses one or more computer-readable media comprising instructions that cause a processor, such as processor 40, to carry out the techniques of the invention. A computer-readable medium includes, but is not limited to, any magnetic or optical storage medium, ROM or EEPROM.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems as described herein. Furthermore, the invention includes embodiments that use techniques to sense physiological parameters in addition to those specifically described herein.

Moreover, the invention is not limited to embodiments that deliver therapy as a function of estimated food intake. The invention includes embodiments that store data in memory concerning food intake, for example, with the data available for later retrieval by the patient or the patient's physician. The invention also includes embodiments that alert the patient of a possible condition that may be affected by food intake. The alert may be delivered by an audible, tactile or radio-controlled alarm. The alerted patient may take appropriate steps to address the condition. These and other embodiments are within the scope of the following claims.

1. A method for sensing food intake by a patient comprising:
measuring a physiological parameter that changes as a function of a quantity of food consumed by the patient; and
estimating the quantity of food consumed by the patient as a function of the measurement.

2. The method of claim 1, wherein measuring the physiological parameter includes measuring a core body temperature of the patient.

3. The method of claim 1, wherein measuring the physiological parameter includes measurement of an electric activity of a gastrointestinal tract of the patient.

4. The method of claim 3, wherein measurement of the electric activity of a gastrointestinal tract comprises measurement of an electric activity of at least one of a stomach, esophagus and intestine of the patient.

5. The method of claim 1, wherein measuring the physiological parameter includes measurement of transabdominal impedance.

6. The method of claim 1, further comprising measuring an activity level of the patient.

7. The method of claim 6, wherein measuring the activity level of the patient comprises measuring physical motion of the patient.

8. The method of claim 6, wherein measuring the activity level of the patient comprises measuring a heart rate of the patient.

9. The method of claim 1, further comprising delivering a therapy to the patient as a function of the estimation.

10. The method of claim 9, wherein delivering the therapy comprises delivering insulin to the patient.

11. The method of claim 9, further comprising measuring an activity level of the patient, wherein delivering the therapy comprises delivering glucagon to the patient as a function of the estimation and a function of the measured activity level.

12. The method of claim 1, further comprising comparing one of the measured physiological parameter and the estimated a quantity of food to a threshold.

13. A system comprising:
a sensor to sense a physiological parameter of a patient that changes as a function of a quantity of food consumed by a patient;
a processor to estimate the quantity of food consumed by the patient as a function of the sensed physiological parameter and to generate a control signal to control a drug delivery system as a function of the estimation; and
the drug delivery system configured to deliver a drug to a body of the patient in response to the control signal.

14. The system of claim 13, wherein the drug delivery system comprises:
a reservoir holding the drug; and
a pump to deliver the drug to the body of the patient by dispensing the drug from the reservoir.

15. The system of claim 13, wherein the system is implanted within the body of the patient.

16. The system of claim 13, wherein the sensor comprises a temperature sensor to sense a core body temperature.

17. The system of claim 13, wherein the sensor comprises at least one electrode to sense electric activity of a gastrointestinal tract.

18. The system of claim 13, wherein the sensor comprises at least one electrode to sense transabdominal impedance.

19. The system of claim 13, wherein the drug comprises at least one of insulin and glucagon.

20. The system of claim 13, wherein the drug delivery system comprises:
a first reservoir holding insulin;
a first pump to deliver the insulin to the body of the patient by dispensing the insulin from the first reservoir;
a second reservoir holding glucagon; and
a second pump to deliver the glucagon to the body of the patient by dispensing the glucagon from the second reservoir.

21. The system of claim 20, wherein the processor is configured to generate a first control signal to control the first pump and a second control signal to control the second pump.

22. The system of claim 13, further comprising memory coupled to the processor to store diet information for the patient.

23. A computer-readable medium comprising instructions that cause a processor to:
estimate a quantity of food consumed by a patient as a function of a measured physiological parameter that changes as a function of the quantity of food consumed by the patient; and
deliver a therapy to the patient as a function of the estimation.

24. The medium of claim 23, wherein the physiological parameter includes a core body temperature of the patient.

25. The medium of claim 23, the instructions further causing the processor to estimate a blood glucose concentration in the patient as a function of the measured physiological parameter and a measured activity level of the patient.
